(19) United States
(12) Patent Application Publication
(10) Pub. No.: US 2018/0344461 A1
(43) Pub. Date: Dec. 6, 2018

(54) VORTEX TRANSDUCTION IMPLANT AND INFLATABLE SENSOR HARBORING PLATFORM

(71) Applicant: Harmony Development Group, Inc., Cornelius, NC (US)

(72) Inventors: John Wilson, Cornelius, NC (US); Christopher Seguin, Norton, MA (US)

(73) Assignee: Harmony Development Group, Inc., Cornelius, NC (US)

(21) Appl. No.: 15/994,690

(22) Filed: May 31, 2018

Related U.S. Application Data
(60) Provisional application No. 62/513,003, filed on May 31, 2017.

(51) Int. Cl. A61F 2/24; A61B 5/02; A61B 5/00; A61B 5/029; A61B 5/145; A61B 5/0215; A61B 8/08; A61B 8/12

(52) U.S. Cl. CPC A61F 2/2487; A61F 2002/482; A61B 5/0031; A61B 5/029; A61B 5/4836; A61B 5/686; A61B 5/6885; A61B 5/14503; A61B 5/14542; A61B 5/14546; A61B 5/02156; A61B 8/0883; A61B 8/12; A61B 8/481; A61B 5/02028

(57) ABSTRACT

An implant system for restoring and improving physiological intracardiac flow in a human heart is provided including an implant for positioning at least partially in the atrium, partially within the atria-ventricular valve, and partially in the ventricle of the human heart and defining a contact surface; a base plate secured to the apex of the heart; a tether assembly connecting the implant to the base plate; and a sensor positioned on at least one of the implant, the base plate, and the tether assembly.

### CROSS-REFERENCE TO RELATED APPLICATIONS

This application claims the benefit under 35 USC § 119(e) of U.S. Provisional Application Ser. No. 62/513,003 filed May 31, 2017, which is hereby incorporated by reference in its entirety.

### TECHNICAL FIELD

The present disclosure relates generally to an implant within a human heart including multiple sensors for monitoring physiological data.

### SUMMARY

An implant system for restoring and improving physiological intracardiac flow, preserving the atrioventricular pressure gradient, and ventricular geometric restoration in a human heart is provided including an implant for positioning at least partially in the atrium, partially within the atrio-ventricular valve, and partially in the ventricle of the human heart and defining a contact surface for directing the intracardiac flow; a base plate secured to the apex of the heart; a tether assembly connecting the implant to the base plate; and a sensor or sensors positioned on at least one of the implant components, the base plate, and the tether or shaft assembly for monitoring cardiac and intracardiac physiological data.

In some embodiments, the sensors are configured to gather, transmit, store, and/or report intracardiac data the implant experiences.

In some embodiments, the sensors are configured to measure cardiac rhythm, blood chemistry levels, and blood oxygen levels. In some embodiments, the sensors are configured to warn of cardiac danger such as pressures, exertion, fatigue, and imminent failure. In some embodiments, the sensors are configured to monitor white cell count, atrial pressures, ventricular pressures, cardiac energy, cardiac forces, transducted forces, flow dynamics and variations, vortical flows and vortical formations, flow vectors, and fluid dynamics.

In some embodiments, the sensors provide feedback to adjust the device. In some embodiments, the implant provides a feedback loop to adjust the resistance of the piston force, energy production, and/or resistance in the dual force configuration.

In some embodiments, the implant and/or control unit has an electronic transmitter to provide feedback outside of the body. The implant can be connected wirelessly to a monitoring platform, and/or to the internet. The implant can be remotely monitored. The implant can report its own status. The control unit, remotely, can control the inflation level of the implant.

### BRIEF DESCRIPTION OF THE DRAWINGS

The objects, features and advantages of the devices, systems, and methods described herein will be apparent from the following description of particular embodiments thereof, as illustrated in the accompanying drawings. The drawings are not necessarily to scale, emphasis instead being placed upon illustrating the principles of the devices, systems, and methods described herein.

FIG. 1 is a cross-sectional diagram illustrating the vortex flow pattern of a healthy human heart.

FIG. 2 is a cross-sectional diagram illustrating the dysfunctional vortex flow pattern of a human heart with pathology.

FIG. 3 is a cross-sectional diagram illustrating the physiology of the chambers of the human heart.

FIG. 4 illustrates the system in accordance with exemplary embodiments of the disclosed subject matter.

FIGS. 5-6 are axial views of the transducting conduit or shaft with the reservoir or balloon deflated in accordance with exemplary embodiments of the disclosed subject matter.

FIG. 7 is a cutaway side view of an implant member in accordance with exemplary embodiments of the disclosed subject matter.

FIG. 8 is a perspective view of a control unit in accordance with exemplary embodiments of the disclosed subject matter.

FIG. 9 is a cross section of a multi lumen tube in accordance with exemplary embodiments of the disclosed subject matter.

FIGS. 10 and 11 are distal end views of the implant member of FIG. 7 disposed in anterior and posterior articulation in accordance with exemplary embodiments of the disclosed subject matter.

FIG. 12 is a side view of the system in accordance with exemplary embodiments of the disclosed subject matter.

FIG. 13 is a top view of the system in accordance with exemplary embodiments of the disclosed subject matter.

FIG. 14 is a cutaway view of an implant member in accordance with exemplary embodiments of the disclosed subject matter.

FIG. 15 is a side view of an implant member in accordance with exemplary embodiments of the disclosed subject matter.

### DETAILED DESCRIPTION

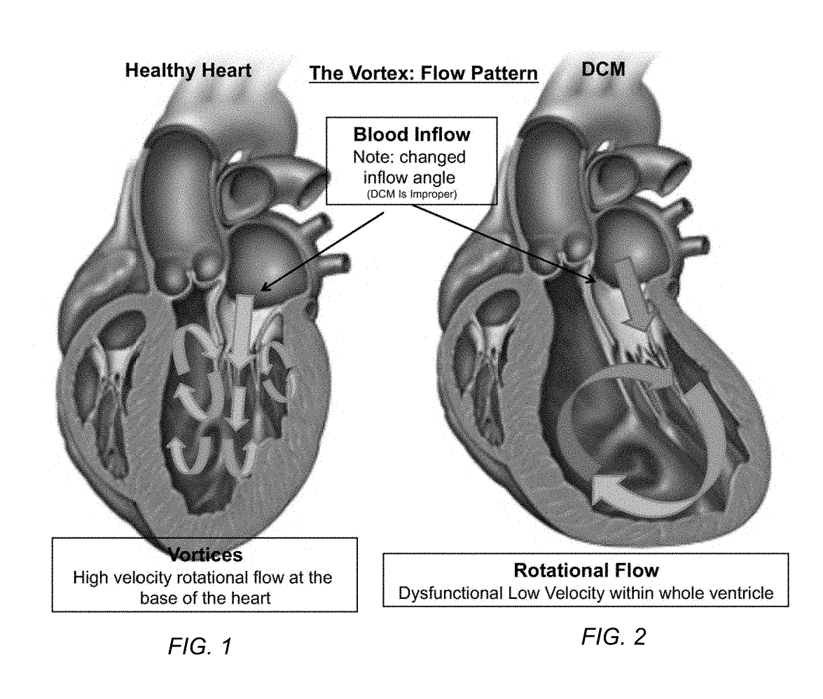

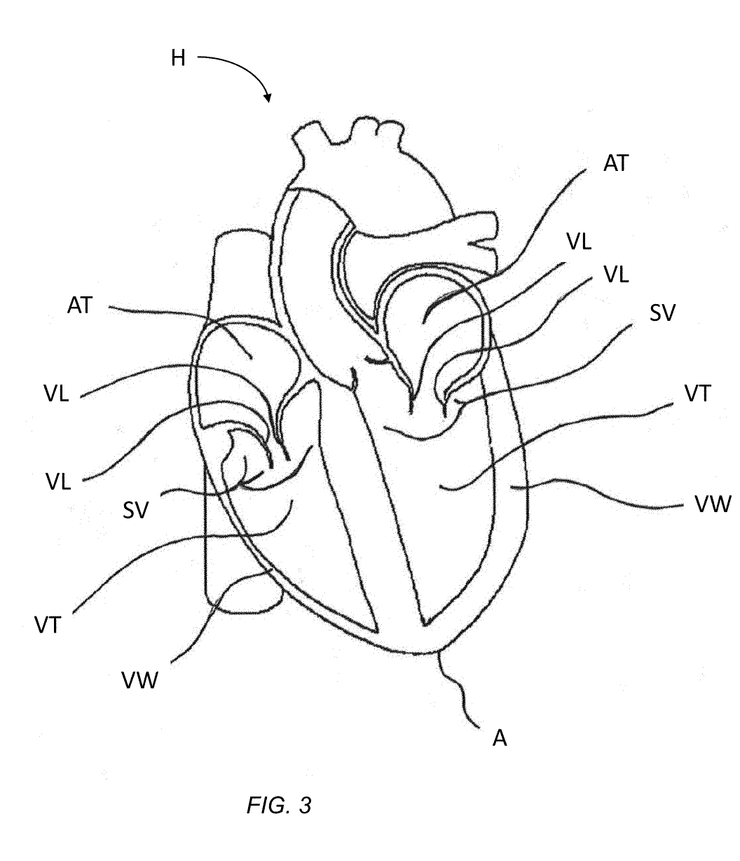

One of the features of healthy heart function is proper physiological intracardiac flow. During ventricular systolic contraction, considerable forces are exerted on the closed atrio-ventricular valve by the atrioventricular pressure gradient. The atrioventricular pressure gradient is defined as the pressure difference (or a pressure differential) that produces or generates an energy and a force within the chambers of the heart that occurs naturally. As the pressure increases in the atrium and the pressure reduces in the ventricle, called the diastolic phase or diastole, blood flows from the higher pressure atrium into the lower pressure ventricle causing the valve leaflets to open and thus allowing the blood to pass through the valve orifice. During the systolic phase or systole, the pressure in the atrium is exceeded by the pressure in the ventricle thereby generating a pressure differential creating an energy and force which, in turn, pushes up and against the valve leaflets causing them to close and seal off the ventricle from the atrial chamber. The atrioventricular pressure gradient is the driving energy and force required to close the valve. These atrioventricular pressure gradient forces are transferred or transducted via the chordae tendinae and papillary muscles into the ventricular wall. There is a resulting valvulo-ventricular wall interaction, which provides and enables the healthy ventricle to maintain structural integrity to maintain healthy elliptical geometry and provides functional support for blood ejection. During ventricular diastole, the ventricular pressure rapidly decreases. The valve opens and blood rushes from the atrium into the ventricle through the valve orifice. The valve leaflets function as a vector or steering mechanism, directing ventricular flow at an angle or vector to create vortical initial spin as illustrated in FIG. 1. Such angle or vector may be due to the asymmetry of the valve leaflets and/or to the different shapes and sizes of the leaflets. A vortex progression results. It is believed that the initial hemodynamic spin begins with inflowing blood, powered by the atrioventricular pressure gradient. On the ventricular side of the valve, the gradient pressure then engages that initial spin such that a vortex is created downstream. The resulting high velocity rotational flow, now a reservoir of kinetic energy within the ventricle is believed significant to proper blood flow velocity and volume through and out of the heart.

FIG. 2 illustrates that under certain conditions, such as a dilated cardiomyopathy (DCM) in which the heart becomes enlarged, the vortex fails to properly form, the elliptical shape is lost, and the papillary muscles displace, and the ventricle is unable to pump blood efficiently. Such conditions are marked by a low velocity flow and poor cardiac output in which the vortices are abnormal or absent and geometric distortion is present creating ventricular dysfunction.

In accordance with the disclosed subject matter, a flow vectoring ‘member’ is implanted in the atrio-ventricular space extending into the ventricle VT of the heart H. It is connected to a tether or shaft anchored at the apex A, and extends through the valve orifice into the atrium AT. When the ventricle VT contracts in systole, the ‘member’ harnesses the valvular and sub valvular energy and force of the atrioventricular pressure gradient by allowing the pressure differential to act on the exposed area of the ‘member’, as the valve leaflets VL and subvalvular apparatus SV grab and pull on the ‘member’, which then transfers or transducts that energy to the therapeutic apical base plate in contact with apex A, via the tethering conduit shaft, in the form of stretching and or torsion utilizing a fluid reservoir. When the ventricle VT relaxes in diastole, releasing the ‘member’, the structure of the flow vectoring ‘member’ intercepts atrial blood and re-vectors it, enabling a changed or altered angle of flow or vector, enabling or assisting the initiation of vortex (i.e., spin) as blood flows off the leaflets and drains into the ventricle VT. By implanting the flow vectoring ‘member’, the normal intracardiac blood flow pattern that is disrupted by pathology or defect and unhealthy ventricular geometry can be restored and geometric distortion repaired via transduction delivered by the therapeutic apical base plate thus restoring the valvulo-ventricular relationship and it's critical ventricular and septal wall interaction by contact.

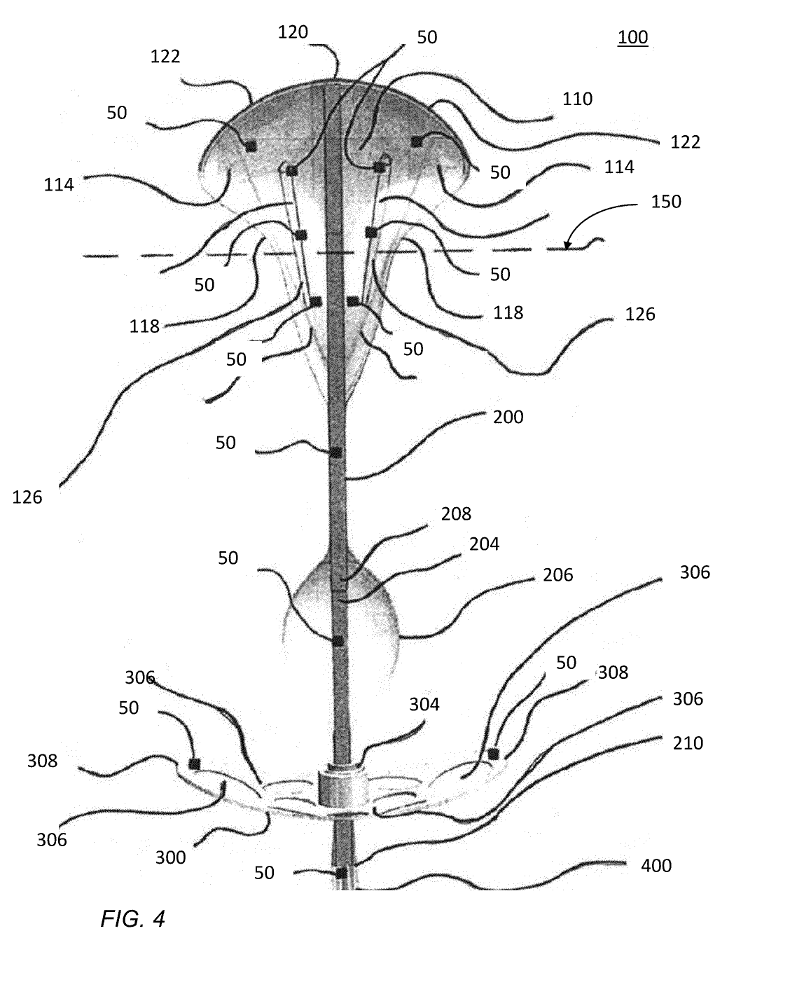

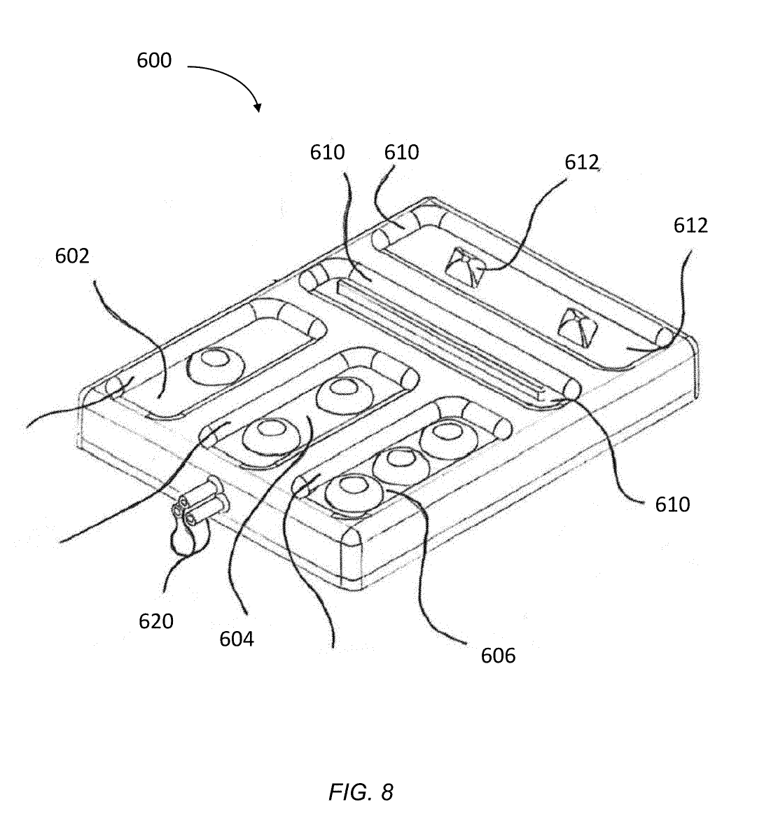

As illustrated in FIG. 4, the implant system 100 in accordance with an exemplary embodiment includes a flow vectoring ‘member’ implant which may be a paddle, or a single, or multi chamber fluid filled member 110 connected to a multi-lumen transducting conduit or shaft 200 (or force transducting tether). The implant 110 is fixed in the intracardiac space of the human heart H, e.g., within the atrium AT, ventricle VT, and/or ventricular leaflets VL. The location of the line of coaptation by the valvular structure is denoted by dotted line 150. The transducting tether or shaft 200 is designed to be fixed to the apex A of the heart H by a therapeutic apical base plate assembly 300. The transducting conduit or shaft 200 is connected to a multi lumen tube 400 after exiting the apex A Multi-lumen tube 400 is connected to a control unit 600, as shown in FIG. 8. Control unit 600 adjusts the device performance via a fluid communicating system when connected to the multi lumen tube 400.

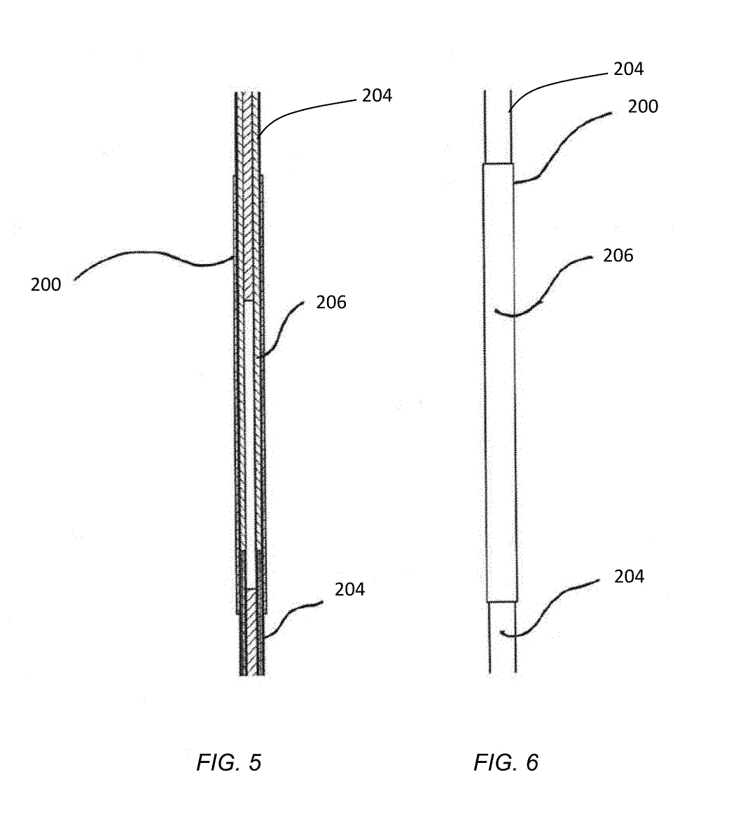

In some embodiments, sensors 50 are provided on one or more locations. For example, sensors 50 are provided on the implant ‘member’ 110, which can be an implant ‘member’ within another implant member and/or a multi chamber fluid filled configuration. The multi lumen transducting fixed shaft 200 may contain sensors 50. As further illustrated in FIGS. 5-6, multi-lumen shaft 200 includes an inner fixed shaft 204, which may contain sensors 50, and an outer axially moving shaft 208, which may contain sensors 50. The outer axially moving shaft 208 supports an integrated inflatable axially adjusting balloon 206, which may contain sensors 50. In some embodiments, the inflatable reservoir or balloon 206 is sized, inflated, or adjusted to accelerate or decelerate inflow blood velocity by removing or offsetting excess ventricular volume facilitating the restoration of proper intracardiac flow. The shaft 200 transitions into a multi lumen tube 400 after exiting the apex A at connection 210.

As illustrated in FIG. 4, shaft 200 includes a sensor 50. In some embodiments, sensors are provided at multiple locations on shaft 200. Shaft 200 is fixed to the apex A of the heart H by an apical base plate 300 have a ball-joint 304, which may contain sensors 50 at various points and that are designed to measure, store, and/or transmit pressure, energy, force, and/or therapeutic transduction data.

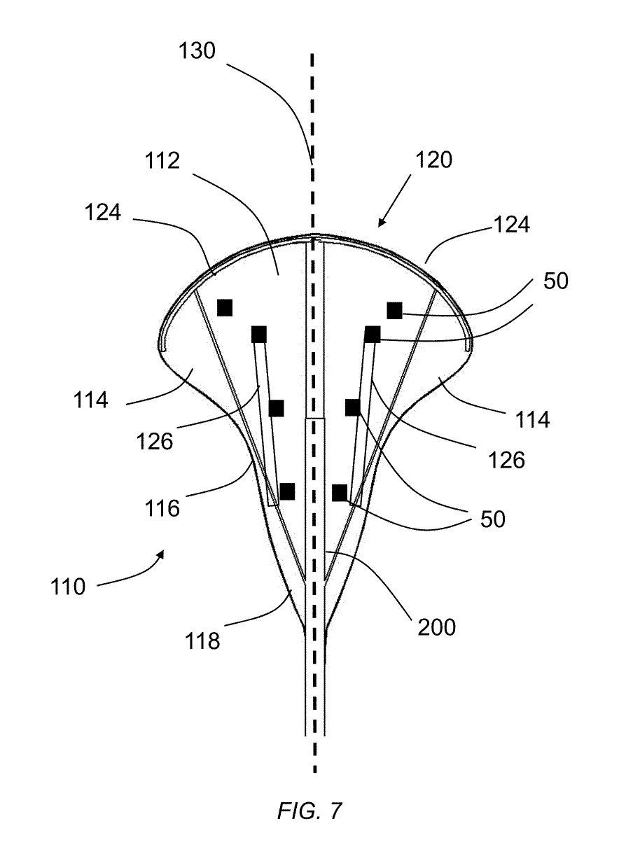

The ‘member’ 110 is illustrated in greater detail in FIG. 7, and is designed with exterior surfaces to intercept, channel, and redirect blood flow from the atrium AT to the ventricle VT. The ‘member’ 110 may have a wider “manta”-shape portion 112 including side wings 114 at distal end portion, that tapers towards a mid-portion 116 and further towards a narrow end portion 118. The “manta”-shape portion 112 is designed to be positioned in the atrium AT, and the mid portion 116 and narrower portion 118 are designed to be positioned through the valve and into the ventricle VT. In some embodiments, ‘member’ 110 includes a lateral (‘wing to wing’) shape support structure, e.g., a skeletal crescent beam 120, fixed to the distal end of the shaft 200 with two force-transducting trusses 124 connecting to the proximal end of the shaft 200 inside of the member 110 to aid in the transduction of captured valvulo-ventricular force and the interception and re-vector of atrial blood flow. The “manta”-shape portion 112 of the ‘member’ 110 intercepts atrial blood and re-vectors it to enhance, facilitate, or restore proper blood flow vector by channeling blood via the flow channel creating ribs 126 and passing blood over and off of the valve leaflets and into the ventricle VT. The ‘member’ 110 captures the native force of the valvular and subvalular structures V as they coapt or ‘grab and pull’ on the member 110 in systole. This capturing action of the ‘member’ 110, its conveyance down the shaft 200, and its connection to the base plate 300 allows for the delivery of this native force and energy, as a ventricular reverse remodeling therapy, to be delivered into the septal wall, the ventricle, and the ventricular free walls resulting in geometric elliptical shape restoration. It is understood that ‘member’ 110 represents one exemplary embodiment of a flow vectoring ‘member’ or other device that may be used in connection with the adjustment assembly described herein. In other embodiments, the shaft may be attached to the distal end of shaft replacement valves, partial prosthetics or valves, regurgitation mitigation devices, and ventricular modification and therapeutic devices. (e.g., replacement valves, partial prosthetic valves, or regurgitation mitigation device). FIGS. 4, 7, 10, 11, 12, 14 and 15 illustrate exemplary locations of the sensor nodes 50 on the implant 110.

With reference to FIG. 7, flow channel creating ribs or ridges 126 are disposed on the surface of ‘member’ 110. Ribs 126 run at angle with respect to the longitudinal axis 130 of the ‘member’ 110, and redirect the intercepted flow of blood onto and off of the valve leaflets, and facilitate proper vector upon entry into the ventricle VT. This hemodynamic re-vector restores the natural physiologic vector thereby facilitating and/or assisting in the restoration of ventricular vortex, critical to physiologic healthy flow. Member 110 includes fluid chamber, in which fluid is added or removed to increase or decrease the member 110 girth or width to increase or decrease the amount of transducted force (by increasing or decreasing the exposed area, meaning in contact, of the implant), vector, volume, and/or velocity and to create crescent-shaped articulation in the wings 114 of member 110.

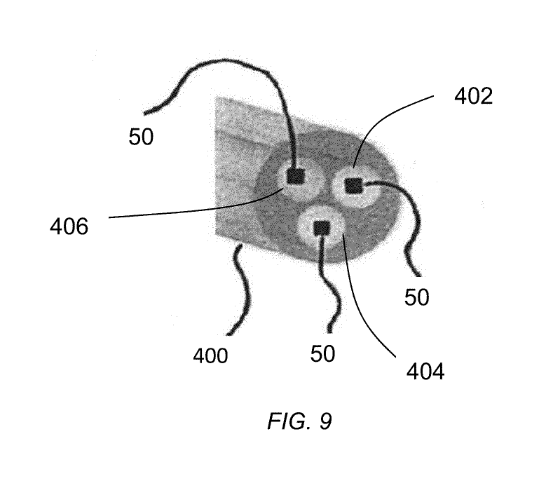

With reference to FIG. 8, the control unit 600, which is implanted in the patient beneath the skin and capable of palpation by the surgeon, is provided to adjust the inflation and/or shape of ‘member’ 110, and the adjustment assembly 800. In some embodiments, control unit 600 is provided with three independent horizontal contained chambers 602, 604 and 606, each identifiable below the skin in some embodiments by palpable protrusions, one palpable protrusion for chamber one 602, two palpable protrusions for chamber two 604, and three palpable protrusions for chamber three 606. Control unit 600 includes vertical chambers 610 for sensor power and/or transmission/component storage, palpable with a ‘square’ protrusion 612 and being elongated in a horizontal directly, orthogonal to the direction of orientation of chambers 602, 604, and 606, with a single connection point 620 for connection to the multi lumen tub 400 (not shown) placing the control unit 600 in communication, via the shaft 200 and its sensors 50. As illustrated in FIG. 9, tube 400, which may contain sensors 50, includes a plurality of lumens 402, 404, and 406 in respective fluid communication with chambers 602, 604 and 606. With continued reference to FIG. 8, the control unit 600 has a needle access pad of silicone and/or ePTFE and/or non-porus and/or any semi-porous material which may allow fibrous tissue in growth (the body's method of preventing infection and facilitating hemostasis).

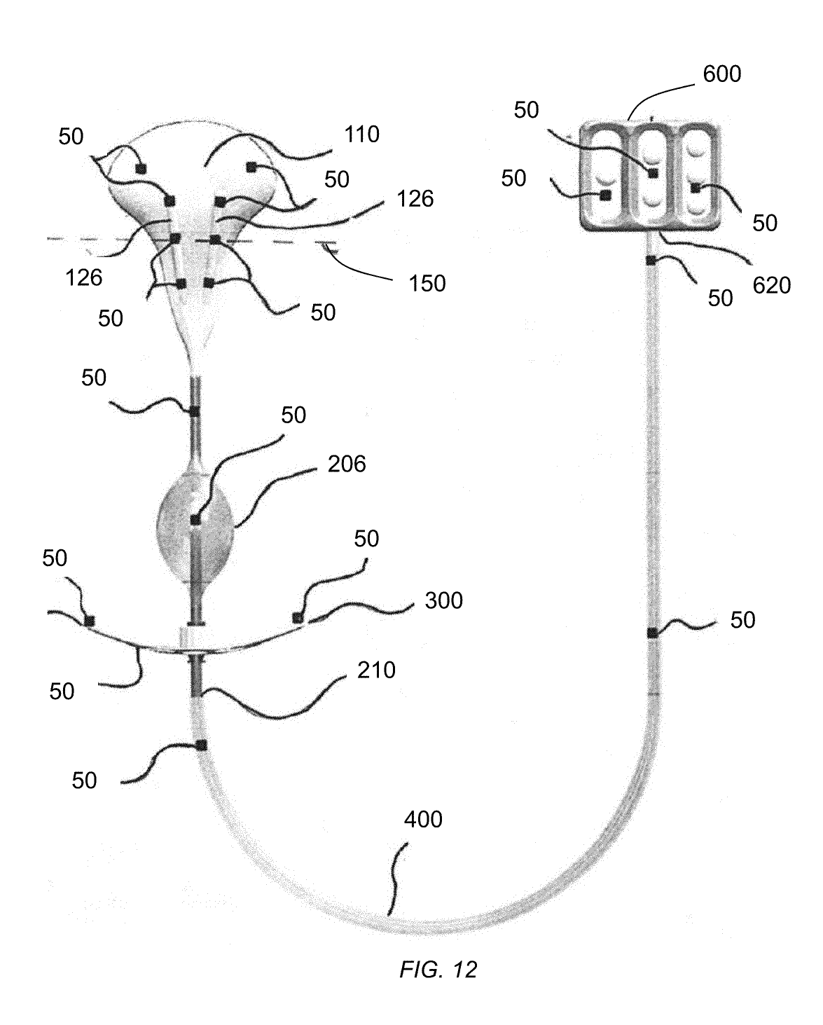

In one embodiment, fluid is introduced or removed with respect to chamber 602 to increase or decrease the girth or width of implant 110. Increasing or decreasing the implant girth alters the vector and adjusts the clinically adjudicated and prescribed amount of force transducted, by increasing or decreasing the exposed area of the implant, to the ventricle VT. The sensors 50, located along 150, on, in, or along 300, and in 200 (as illustrated in FIGS. 4 and 12) provide real-time data with respect to atrial outflow and ventricular inflow hemodynamics and allows the system to provide feedback to adjust the flow vector by increasing/decreasing the amount of fluid contained within implant 110, thereby increasing/decreasing the girth or width of implant 110.

In one embodiment, fluid is introduced or removed with respect to chamber 604 to increase or decrease fluid into axial adjusting balloon 206, thereby adjusting the longitudinal axial position of shaft 204 and thus the longitudinal position of implant 110 with respect to the ventricle VT, atrium AT, valvular leaflets VL and the line of coaptation 150 as reverse (positive) re-modeling occurs. The sensors 50, located along 126 and on or near 150, provide real-time data with respect to proper axial positioning to maximize the capture and delivery of native energy and forces to the septal wall, the ventricle, and the ventricular free walls and allows the system to provide feedback to adjust axially by increasing/decreasing the amount of fluid inside balloon 206, thereby adjusting the longitudinal position of implant 110.

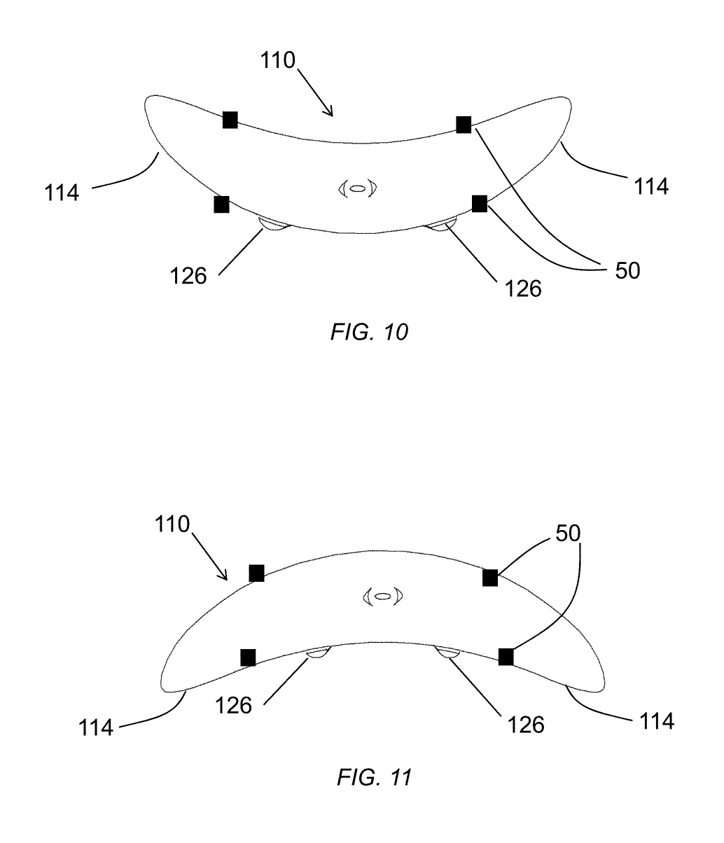

In one embodiment, fluid is introduced or removed with respect to chamber 606 to create crescent shaped articulation in the wings 114 of implant 110, either anterior (FIG. 10) or posterior (FIG. 11), to better intercept, guide, and vector atrial blood by introducing fluid into the wing chambers via the skeletal crescent beam 122 via a lumen in shaft 200. The sensors 50 located near, at, or on the wing bladders provide real-time data with respect to hemodynamic pattern, velocity, and flow and allows the system to provide feedback to adjust articulation, either anterior or posterior in scope, by increasing/decreasing volume of fluid into or out of the implant wings, thereby adjusting crescent-shaped articulation of implant 110.

In one embodiment, chambers 610, or sealed vertical compartments, house a power source, transmitting source, and/or a data storage source for sensoring nodes 50 implanted within the implant system 100 itself. As such, the implant system 100 serves as a housing platform for the sensoring nodes 50. One or more lumens of the connecting multi-lumen connecting tube 400 connect the power source in chambers 610 with the sensoring nodes 50. Sensoring nodes 50 are devices, e.g., transducers, or other components which identify, detect, and electronically report glucose, blood chemistry, any diagnostic values, pressure, pressure changes, velocities, and velocity changes, etc., and may be embedded in the components of the system and/or its platform for any diagnostic detection, the detection, restoration, and management of vortex, vorticial flow, and assist in providing transduction therapy, e.g., as it applies to the ventricle VT and ventricular walls VW.

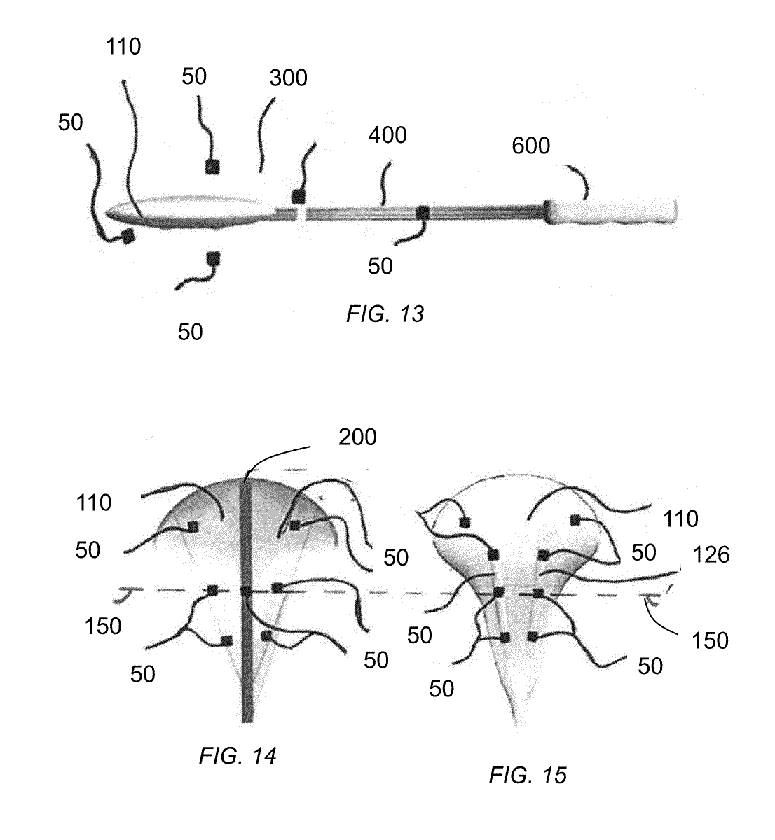

The fixed apical base plate 300, with round oval cutouts 306 to allow fibrous tissue in-growth for long term security, pulls the apex A upward in systole and releases the apex A in diastole and, in conjunction with the elongated 308 therapeutic extensions of the apical base 300 plate extending up the sides of the ventricle VT, impart by contact, specific shape, and fixation this transducted energy, with the ability to be monitored by sensoring technology 50 may be adjusted to individual pathology and/or patient specific requirements, intra and/or post operatively, via the control unit 600, delivering into said ventricle VT reduced or amplified energy and force based on specific clinical need, inducing a physiologic response by replacing the lost valvulo-ventricular interaction required to maintain healthy geometric shape of the septal, ventricle, ventricular structures, and the ventricular wall VW. FIGS. 4, 12 and 13 illustrate exemplary locations of the sensor nodes 50 on the apical base plate 300.

It will be appreciated that the methods and systems described above are set forth by way of example and not of limitation. Numerous variations, additions, omissions, and other modifications will be apparent to one of ordinary skill in the art. Thus, while particular embodiments have been shown and described, it will be apparent to those skilled in the art that various changes and modifications in form and details may be made therein without departing from the spirit and scope of this disclosure and are intended to form a part of the disclosure as defined by the following claims, which are to be interpreted in the broadest sense allowable by law.

What is claimed is:

1. An inflatable intracardiac system for monitoring and altering physiological blood flow in a human heart comprising:
an implant configured for positioning at least partially in the atrium, partially within the atria-ventricular valve, and partially in the ventricle of the human heart and defining a contact surface;
a base plate configured to be secured to the heart;
a tether/conduit assembly connecting the implant to the base plate; and
a sensor positioned on at least one component of the implant, or the base plate, and or the tether assembly; and
a control unit to house sensoring components, power, data storage, and adjust fluid volumes.

2. The intracardiac system of claim 1, wherein the sensor comprises a transducer, or a diagnostic surveillance system positioned on or in the implant.

3. The intracardiac system of any one of the preceding claims, wherein the sensor is configured to gather, transmit, store, and/or report intracardiac data the implant experiences.

4. The intracardiac system of any one of the preceding claims, wherein the sensor is configured for positioning on the atrial end thereof to measure data, gather data, or transmit data on the atrial and/or valvulo-ventricular action, flows and/or forces.

5. The intracardiac system of any one of the preceding claims, wherein the sensor is positioned on the ventricular end thereof to measure data, gather data, or transmit data on the ventricular and/or valvulo-ventricular action, flows, and/or forces.

6. The intracardiac system of any one of the preceding claims, wherein the sensor is positioned on the apical base plate and/or tether assembly to measure data, gather data, or transmit data on the ventricular structure, the ventricular walls, and/or valvulo-ventricular action, flows, and/or forces.

7. The intracardiac system of any one of the preceding claims, wherein the tether assembly comprises a conduit shaft, and wherein the sensor is positioned on the length of the shaft to measure data, gather data, or transmit data on the ventricle, the hemodynamic flows, and/or the valvulo-ventricular action and/or forces.

8. The intracardiac system of any one of the preceding claims, wherein the sensor measures and/or monitors cardiac rhythm by monitoring pressure cycles in the implant.

9. The intracardiac system of any one of the preceding claims, wherein the sensor measures and/or monitors blood chemistry levels from a port in the tether and or conduit.

10. The intracardiac system of any one of the preceding claims, wherein the sensor measures and/or monitors cardiac systems, function, and exertion by measuring the forces transferred to the baseplate.

11. The intracardiac system of any one of the preceding claims, wherein the sensor measures and/or monitors blood oxygen levels by utilizing a break in the multi-lumen tubing to read the blood flowing passed.

12. The intracardiac system of any one of the preceding claims, wherein the sensor warns, measures, and/or monitors for blood infection by utilizing a port in the multi-lumen tubing and a sensor in the lumen or control unit.

13. The intracardiac system of any one of the preceding claims, wherein the sensor measures and/or monitors white cell count.

14. The intracardiac system of any one of the preceding claims, wherein the sensor measures and/or monitors atrial pressures.

15. The intracardiac system of any one of the preceding claims, wherein the sensor measures and/or monitors ventricular pressures.

16. The intracardiac system of any one of the preceding claims, wherein the sensor measures and/or monitors cardiac energy from the forces received from tether and/or implant.

17. The intracardiac system of any one of the preceding claims, wherein the sensor measures and/or monitors cardiac forces.

18. The intracardiac system of any one of the preceding claims, wherein the sensor measures and/or monitors transducted, captured, harnessed, and delivered forces.

19. The intracardiac system of any one of the preceding claims, wherein the sensor measures and/or monitors flow dynamics and variations.

20. The intracardiac system of any one of the preceding claims, wherein the sensor measures and/or monitors vortical flows and vortical formations with measurements of deflection of the tether and/or conduit.

21. The intracardiac system of any one of the preceding claims, wherein the sensor measures and/or monitors flow vectors.

22. The intracardiac system of any one of the preceding claims, wherein the ultrasonic sensor measures and/or monitors fluid dynamics.

23. The intracardiac system of any one of the preceding claims, wherein the implant with the multi-lumen tubing has ports linked to specific sensors to measure and/or monitor INR levels, anticoagulation levels, and/or anti-clotting or thrombolytic medication levels.

24. The intracardiac system of any one of the preceding claims, wherein the implant has hemodynamic communication via a lumen or a separate tube connecting to a chamber in the control unit for a direct intracardiac drug injection.

25. The intracardiac system of any one of the preceding claims, wherein the sensors measure, monitor, and provide feedback to adjust the device.

26. The intracardiac system of any one of the preceding claims, wherein the implant has an electronic transmitter to deliver and provide feedback outside of the body.

27. The intracardiac system of any one of the preceding claims, wherein the implant is connected wirelessly to a monitoring platform, and/or to the internet via a transmitter or another wireless component or device.

28. The intracardiac system of any one of the preceding claims, wherein the implant is remotely monitored.

29. The intracardiac system of any one of the preceding claims, wherein the implant reports its own status from the control unit.

30. The intracardiac system of any one of the preceding claims, wherein the implant controls the inflation level of the implant by inflating or deflating from a remote fluid reservoir to establish a known pressure or fill level to calibrate and/or recalibrate sensors.

31. The intracardiac system of any one of the preceding claims, wherein the implant delivers and provides a feedback loop to adjust the resistance of the piston force, energy production, and/or resistance in any configuration.

32. The intracardiac system of any one of the preceding claims, wherein the implant is a harboring platform for another specific device implanted within the host device.

33. The intracardiac system of any one of the preceding claims, wherein the implant is an inflatable intracardiac implantable device that is a hosting harbor for sensoring implants and/or specific sensors.

34. The intracardiac system of any one of the preceding claims, wherein the implant is an inflatable intracardiac implantable device harbor for sensoring nodes, diagnostic sensors, and/or monitoring devices in which the implant itself may be inflated to a known pressure and the sensoring devices themselves may be calibrated based on that known pressure while remaining implanted within the cardiac structure itself from outside the body.

35. The intracardiac system of any one of the preceding claims, wherein the shaft or tether has stress sensors along its length to monitor force transferred from the end effector to the baseplate, and shaft deflection during ventricular filling.

36. The intracardiac system of any one of the preceding claims, wherein the control unit can remotely monitor with internal pressure sensors the pressures in the device via the fluid connection.

37. The intracardiac system of any one of the preceding claims, wherein the control unit can transmit electrical signals and/or communications to a needle or a connected apparatus that is inserted into or onto one of the needle access areas on the control unit.

38. The intracardiac system of any one of the preceding claims, wherein the control unit has features for palpation and locating and identifying it subcutaneously.

39. The intracardiac system of any one of the preceding claims, wherein the control unit has an internal power source.

40. The intracardiac system of any one of the preceding claims, wherein the control unit may capture and use energy from the pressure gradient to power the sensors.

41. The intracardiac system of any one of the preceding claims, wherein the control unit may be charged while implanted subcutaneously.

42. The intracardiac system of any one of the preceding claims, wherein the control unit can be charged, either with fluid or with power, by inserting special needles into two needle access sites on the control unit.

43. The intracardiac system of any one of the preceding claims, wherein the stress sensors may provide the control unit information specific to measuring on the amount of force being transferred through the shaft and into the base plate and from the base plate into the structures in contact.

44. The intracardiac system of any one of the preceding claims, wherein the conduit shaft transitions to multi-lumen tubing and is purposed for fluid transfer, contains wires and/or fiber optic cable for information transfer, and transfers force from the implant to the base plate.

45. The intracardiac system of any one of the preceding claims, wherein the multi-lumen tether/conduit includes ports open to the intracardiac blood to take samples.

46. The intracardiac system of any one of the preceding claims, wherein the multi-lumen tether/conduit has a light source at one end and a reader at the other end of the same lumen.

47. The intracardiac system of any one of the preceding claims, wherein the lumen has a sealed break in it to allow blood to flow passed the sensor.

48. The intracardiac system of any one of the preceding claims, wherein the intracardiac platform of any one of the preceding claims, in which the base plate has a ball joint.

49. The intracardiac system of any one of the preceding claims, wherein the ball joint of preceding claims has a position and rotation sensor on it monitor tether movement.

50. The intracardiac system of any one of the preceding claims, wherein the inflatable intracardiac platform includes multi lumen tubing containing an intracardiac echocardiography transmitter and reader in one of the lumens.

51. The intracardiac system of any one of the preceding claims, wherein the inflatable intracardiac platform monitors blood flow and cardiac function.

52. The intracardiac system of any one of the preceding claims, wherein the fluid in the implant exhibits echogenic properties to highlight its own features and or function.
